(19) United States
(12) Patent Application Publication
(10) Pub. No.: US 2003/0225148 A1
(43) Pub. Date: Dec. 4, 2003

(54) Biological methods of use of 4-amino-3-mercapto-triazoles

(76) Inventors: Ned H. Heindel, Easton, PA (US); Jeffrey D. Laskin, Piscataway, NJ (US); Diane E. Heck, Rumson, NJ (US); Christophe Guillon, Bethlehem, PA (US); Robert D. Rapp, Reading, PA (US)

Correspondence Address: PERKINS COIE LLP, POST OFFICE BOX 1208, SEATTLE, WA, 98111-1208, US

(21) Appl. No.: 10/245,102

(22) Filed: Sep. 17, 2002

Related U.S. Application Data
(63) Continuation of application No. 09/790,330, filed on Feb. 21, 2001.

(30) Foreign Application Priority Data
Jul. 30, 1991 (DE) P 41 25 611.5
Mar. 25, 1992 (DE) P 42 10 165.4

(51) Int. Cl. A61K 31/4196; A61K 7/22

(52) U.S. Cl. 514/384; 424/54

(57) ABSTRACT

This invention claims the utility of two structural variants of functionalized 4-amino-3-mercapto-1,2,4-triazoles as inhibitors of nitric oxide synthase (NOS) and as inhibitors of malignant cell growth. This fundamental molecular construct operates as a heterocyclic mimic of the open-chain N-aminoarginines (or N-aminoguanidines) previously established as NOS inhibitors. A convenient bioassay method, using PAM 212 keratinocytes for detection and quantification of relative NOS inhibition potential in a series of candidate drugs, is described as is a bioassay for growth inhibition.

### CROSS-REFERENCE TO RELATED APPLICATIONS

This application is a continuation of U.S. patent application Ser. No. 09/790,330, filed Feb. 21, 2001, which is the U.S. national phase of International Application No. PCT/US99/19146, filed Aug. 21, 1999, which claims priority to U.S. Provisional Patent Application No. 60/242,160, filed Aug. 21, 1998, the disclosures of which are incorporated by reference herein in their entirety.

### BACKGROUND OF THE INVENTION

From a clinical perspective it is very clear that in vivo pharmacological manipulation of nitric oxide (NO) production will be of considerable therapeutic value. The list of nitric oxide synthase mediated diseases grows longer every year but the broad classes of dysfunctions includes many gastrointestinal motility problems, inflammatory states, and neurodegenerative disorders. A partial array of specific medical circumstances which appear to be certainly associated with NOS are: sunburn, rheumatoid arthritis, ulcerative colitis, Crohn's disease, lupus, septic and toxic shock, asthma, hypertension, myocarditis, diabetes, and many autoiummune and respiratory problems (Macdonald-1996).

Now that it is known that the various isoforms of NOS utilize the arginine to citrulline deamination as the route to NO, many therapeutic drugs have been designed to target that pathway (Kerwin-1994). With a wide variety of N-gamma-substituted arginines identified as inhibitors of NOS bearing such pendant gamma residues as nitro, amino, and even alkyl, and with the observation that some heterocyclic triazole systems appear to mimic the guanidino portion of arginine (Buchmuller-Rouiller-1992), we proposed the use of planar, fused-ring bio-isosteric models of arginine as new candidate classes of NOS inhibitors.

While 1,2,4-triazoles do have an abundant patent literature as useful agriculturals and even as human therapeutics (Camden-1998, Tomioka-1998, and Reitz-1997), the specific prior art on the 4-amino-1,2,4-triazoles indicated they did not possess inhibitory activity against nitric oxide synthase (NOS) (Buchmuller-Rouiller-1992). We have found, however, significant NOS-inhibitory activity in that 4-amino-1,2,4-triazole family bearing a pendant 3-mercapto moiety. Furthermore, active NOS inhibitors were also found and in several of the N- or S-functionalized derivatives of these 4-amino-3-mercapto-(4H)-1,2,4-triazoles, see FIG. 1.

We believe these heterocyclic candidate therapeutics are functioning as cyclic biological isosteres of the N-aminoguanidines previously shown to possess NOS inhibition (Macdonald-1996).

We also report herein the utility of Classes VII and X as anticancer therapeutics active against a broad array of malignant cell types, a pharmacology without precedent in these families. Camden has reported that certain specially substituted N-alkyl-1,2,4-triazoles do display antineoplastic activity (Camden-1998). None of our anticancer families is N-alkyl substituted.

### DETAILED DESCRIPTION OF THE INVENTION

This invention is directed to a pharmacologically acceptable composition for inhibiting nitric oxide synthase (NOS) in a mammal and inhibition of cancer cell growth. The composition includes members of Classes VII to X and a pharmaceutically acceptable carrier, with the substituted triazole derivative present in the composition in an effective amount to inhibit NOS or cancer cell growth in the mammal. The invention is also directed to a method of inhibiting NOS in a mammal, which includes the step of administering to the mammal the triazole derivative in pure form or in a pharmaceutically acceptable carrier. Suitable derivatives for use in the composition or method can be synthesized. Those members of Classes VII to X possessing basic nitrogen atoms may be utilized as a pharmaceutically acceptable salt thereof. This invention discloses that these triazole derivatives are inhibitors of NOS and cancer cell growth. Additional NOS isoform-specific inhibitors can be generated from derivatives using chemical manipulations.

Classes VII to X, in pure form or in a pharmacologically acceptable carrier, will find benefit in treating conditions and disorders where there is an advantage in inhibiting the nitric oxide synthase enzyme. Said inhibitors may be used in treat circulatory shock; they may also be beneficial for patients receiving therapy with cytokines such as TNF, IL-1 and IL-2 or therapy with cytokine-inducing agents, or as a short-term immunosuppression in transplant therapy. In addition, the derivatives may be useful to inhibit NO synthesis in patients suffering from inflammatory conditions in which an excess of NO contributes to the pathophysiology of the condition, such as inflammatory bowel disease, adult respiratory distress syndrome and myocarditis, for example. Nitric oxide has been found to play a role in inflammatory responses resulting from pharmacologic agents such as acetaminophen and other nonsteroidal antiinflammatory drugs as well as in injury resulting from exposure to environmental toxins.

Nitric oxide inhibitors may be effective in alleviating drug interactions, toxicity from environmental contaminants and war gases. There is also evidence that an NO synthase enzyme may be involved in the pathophysiology of autoimmune and/or inflammatory conditions such as arthritis, rheumatoid arthritis and systemic lupus erythematosus and in insulin-dependent diabetes mellitus, and therefore, the triazole derivatives described herein may prove helpful in treating these conditions. Furthermore, it is now clear that there are a number of additional inflammatory and non-inflammatory diseases that are associated with NO over-production. Examples of such physiological disorders include: inflammatory bowel diseases such as ileitis, ulcerative colitis and Crohn's disease; inflammatory lung disorders such as asthma and chronic obstructive airway disease; inflammatory disorders of the eye including corneal dystrophy, trachoma and onchocerciasis; chronic inflammatory disorders of the gum including periodontitis; chronic inflammatory disorders of the joints including arthritis and osteoarthritis, tuberculosis, leprosy and nephrosis; disorders of the skin including sclerodermatitis, psoriasis and eczema; inflammatory diseases of the central nervous system, including chronic demyelinating diseases such as multiple sclerosis, muscular dystrophy, dementia including AIDS-related neurodegeneration and Alzheimer's disease, Parkinson's disease, amyelotrophic lateral sclerosis, encephalomyelitis and viral or autoimmune encephalitis; autoimmune diseases including immune-complex vasculitis, systemic lupus and erythematodes; and disease of the heart including ischemic heart disease and cardiomyopathy.

Additional disease that may benefit from the use of these 4-amino-3-mercapto-1,2,4-triazole derivatives include adrenal insufficiency; hypercholesterolemia; atherosclerosis; bone disease associated with increased bone resorption, e.g. osteoporosis, pre-eclampsia, eclampsia, uremic complications; chronic liver failure, non-inflammatory diseases of the central nervous system including stroke and cerebral ischemia; and various forms of cancer.

Pharmaceutical formulations of the 4-amino-3-mercapto-1,2,4-triazole analogs may include those suitable for oral, rectal, nasal, topical (including buccal and sub-lingual), eye drops, ear drops, vaginal or parenteral (including intramuscular, sub-cutaneous and intravenous) administration, or for administration by inhalation or insufflation. The formulations may, where appropriate, be conveniently presented in discrete dosage units and may be prepared by any of the methods well known in the art of pharmacy. All methods include the steps of bringing into association the active compound with liquid carriers or finely divided solid carriers or both, and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion; or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example, saline, water-for-injection, immediately prior to use.

Extemporaneous injection solutions and suspensions may be prepared from sterile powders, granules, and tablets of the kind previously described.

Formulations for rectal administration may be presented as a suppository with the usual carriers such as cocoa butter or polyethylene glycol. Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation the active ingredient is conveniently delivered from an insufflator, nebulizer pressurized packs or other convenient means of delivering an aerosol spray. Pressurized packs may comprise a suitable propellant such as dichlorodifluoromethane, carbon dioxide or other suitable gas. In the case of a pressurized aerosol, the dosage unit may be determined by providing a valve to deliver a metered amount.

Alternatively, for administration by inhalation or insufflation, the active ingredient may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

The compositions useful in the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the invention, stabilizers, preservatives, excipients, and the like. The preferred lipids are the phospholipids and the phosphatidyl cholines (lecithins), both natural and synthetic. Methods to form liposomes are known in the art (see e.g., Prescott, E., Meth. Cell Biol. 14:33 (1976)).

Other pharmaceutically acceptable carrier includes, but is not limited to, a non-toxic solid, semisolid or liquid filler, diluent, encapsulating material or formulation auxiliary of any type, including but not limited to ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Solid pharmaceutical excipients include, but are not limited to, starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients can be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Methods of preparing various pharmaceutical compositions with a certain amount of active ingredient are known, or will be apparent in light of this disclosure, to those skilled in this art. Other suitable pharmaceutical excipients and their formulations are described in Remington's Pharmaceutical Sciences, edited by E. W. Martin, Mack Publishing Company, 19th ed. (1995).

The active ingredient may also be used in combination with other therapeutic agents, for example, anti-inflammatory agents, particularly non-steroidal anti-inflammatory drugs (NSAIDs), and vasodilator prostaglandins.

It should be understood that in addition to the ingredients particularly mentioned above, the formulations of this invention may include other agents conventional in the art having regard to the type of formulation in question, for example those suitable for oral administration may include flavoring agents. Preferred unit dosage formulations are those containing an effective dose, as recited below, or an appropriate fraction thereof, of the active ingredient.

One of ordinary skill in the art will appreciate that pharmaceutically effective amounts of the NOS inhibitor can be determined empirically and can be employed in pure form or, where such forms exist, in pharmaceutically acceptable salt, ester or prodrug form. The agents can be administered to a patient as pharmaceutical compositions in combination with one or more pharmaceutically acceptable excipients. It will be understood that, when administered to, for example, a human patient, the total daily usage of the agents or composition of the present invention will be decided within the scope of sound medical judgement by the attending physician. The specific therapeutically effective dose level for any particular patient will depend upon a variety of factors: the type and degree of the cellular response to be achieved; activity of the specific agent or composition employed; the specific agents or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

For each of the aforementioned conditions, the NOS inhibitors of the invention may be administered orally or via injection at a dose of from 1 to 250 mg/kg per day. The dose range for adult humans is generally from 50 mg to 17.5 g/day and preferably 150 mg to 3 g/day. Tablets or other forms of presentation provided in discrete units may conveniently contain an amount which is effective at such dosage or as a multiple of the same, for instance, units containing 5 mg to 500 mg, usually around 100 mg to 500 mg.

The pharmaceutical composition preferably is administered orally or by injection (intravenous or subcutaneous), and the precise amount administered to a patient will be the responsibility of the attending physician. However, the dose employed will depend upon a number of factors, including the age and sex of the patient, the precise disorder being treated, and its severity. Also the route of administration may vary depending upon the condition and its severity.

The pharmaceutical composition may be used for treating mammals including humans.

Glossary

Pharmacologically Acceptable Composition: An amount of the relevant compound useful for treatment of a patient.

Mammal: Refers to any animal classified as a mammal, including humans, domestic and farm animals, and zoo, sports and pet companion animals such as a household pet and other domesticated animal such as, but not limited to, cattle, sheep, ferrets, swine, horses, poultry, rabbits, goats, dogs, cats, and the like. Preferred companion animals are dogs and cats. Preferably, the mammal is human.

Nitric Oxide Synthase Mediated Diseases: Disorders mediated by insufficient or excessive release of nitric oxide.

Patient: a mammal, preferably a human, in need of treatment of a condition, disorder or disease.

Treat and Treatment: Refer to both therapeutic treatment and prophylactic or preventative measures, wherein the object is to prevent or slow down (lessen) an undesired physiological condition, disorder or disease or obtain beneficial or desired clinical results. For purposes of this invention, beneficial or desired clinical results include, but are not limited to, alleviation of symptoms; diminishment of extent of condition, disorder or disease; stabilized (i.e. not worsening) state of condition, disorder or disease; delay or slowing of condition, disorder, or disease progression; amelioration of the condition, disorder or disease state, remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

Inhibitor: includes but is not limited to, any suitable molecule, compound, protein or fragment thereof, nucleic acid, formulation or substance that can regulate NOS activity in such a way that NOS is decreased. The inhibitor can include, but is not limited to the specifically identified Classes VII and X.

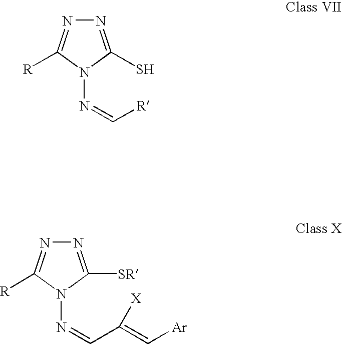

Alkyl: refers to saturated aliphatic groups including straight-chain, branched-chain and cyclic groups having the number of carbon atoms specified, or if no number is specified, having up to 12 carbon atoms. The term “cycloalkyl” as used herein refers to a mono-, bi-, or tricyclic aliphatic ring having 3 to 14 carbon atoms and preferably 3 to 7 carbon atoms.

Aryl: Refers to and which is included with the term “carbocyclic ring structure” refers to an unsubstituted or substituted aromatic ring, substituted with one, two or three substituents selected from lower alkoxy, lower alkyl, lower alkylamino, hydroxy, halogen, cyano, hydroxyl, mercapto, nitro, thioalkoxy, carboxaldehyde, carboxyl, carboalkoxy and carboxamide, including but not limited to carbocyclic aryl, heterocyclic aryl, and biaryl groups and the like, all of which may be optionally substituted. Preferred aryl groups include phenyl, halophenyl, loweralkylphenyl, napthyl, biphenyl, phenanthrenyl and naphthacenyl.

As used herein, the term “heterocyclic ring” or “heterocyclic ring system” is intended to mean a substituted or unsubstituted member selected from the group consisting of stable monocyclic ring having from 5-7 members in the ring itself and having from 1 to 4 hetero ring atoms selected from the group consisting of N, O and S; a stable bicyclic ring structure having a total of from 7 to 12 atoms in the two rings wherein at least one of the two rings has from 1 to 4 hetero atoms selected from N, O and S, including bicyclic ring structures wherein any of the described stable monocyclic heterocyclic rings is fused to a hexane or benzene ring; and a stable tricyclic heterocyclic ring structure having a total of from 10 to 16 atoms in the three rings wherein at least one of the three rings has from 1 to 4 hetero atoms selected from the group consisting of N, O and S. Any nitrogen and sulfur atoms present in a heterocyclic ring of such a heterocyclic ring structure may be oxidized. Unless indicated otherwise the terms “heterocyclic ring” or “heterocyclic ring system” include aromatic rings, as well as non-aromatic rings which can be saturated, partially saturated or fully saturated non-aromatic rings. Also, unless indicated otherwise the term “heterocyclic ring system” includes ring structures wherein all of the rings contain at least one hetero atom as well as structures having less than all of the rings in the ring structure containing at least one hetero atom, for example bicyclic ring structures wherein one ring is a benzene ring and one of the rings has one or more hetero atoms are included within the term “heterocyclic ring systems” as well as bicyclic ring structures wherein each of the two rings has at least one hetero atom. Moreover, the ring structures described herein may be attached to one or more indicated pendant groups via any hetero atom or carbon atom which results in a stable structure. Further, the term “substituted” means that one or more of the hydrogen atoms on the ring carbon atom(s) or nitrogen atom(s) of the each of the rings in the ring structures described herein may be replaced by one or more of the indicated substituents if such replacement(s) would result in a stable compound. Nitrogen atoms in a ring structure may be quaternized, but such compounds are specifically indicated or are included within the term “a pharmaceutically acceptable salt” for a particular compound. When the total number of O and S atoms in a single heterocyclic ring is greater than 1, it is preferred that such atoms not be adjacent to one another. Preferably, there are no more than one O or S ring atoms in the same ring of a given heterocyclic ring structure.

Examples of monocyclic and bicyclic heterocyclic ring systems, in alphabetical order, are acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl (benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyroazolidinyl, pyrazolinyl, pyrazolyl, pyridazinyl, pryidooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl. Preferred heterocyclic ring structures include, but are not limited to, pyridinyl, furanyl, thienyl, pyrrolyl, pyrazolyl, pyrrolidinyl, imidazolyl, indolyl, benzimidazolyl, 1H-indazolyl, oxazolinyl, or isatinoyl. Also included are fused ring and spiro compounds containing, for example, the above heterocyclic ring structures.

Halo or halogen: refer to Cl, Br, F or I substituents. The term “haloalkyl”, and the like, refer to an aliphatic carbon radicals having at least one hydrogen atom replaced by a Cl, Br, F or I atom, including mixtures of different halo atoms. Trihaloalkyl includes trifluoromethyl and the like as preferred radicals, for example.

### EXAMPLES

The following examples are provided by way of illustration, and are not intended to limit the scope of the invention.

### Example 1

### Methods for Assessing Inhibitors of Nitric Oxide Synthase

Compounds were assayed by examining their ability to inhibit nitric oxide production from PAM 212 keratinocytes stimulated to produce nitric oxide synthase with the cytokine gamma interferon as described by Heck et al. (1992) and DeGeorge et al. (1997).

A typical response curve for VIIa, R=2-thienyl, R′=CH═CH-Ph is shown in FIG. 2.

### Example 2

### Results of NOS Inhibitors

Members of all triazole and fused-ring triazole families (VII and X) displayed NOS-inhibiting activity in this assay. Typical values as IC₅₀'s obtained from the testing and graphical analysis described above were:

| | | |
|---|---|---|
| (VIIa, R = 2-thienyl, R′ = —CH═CH—Ph) | 35 | uM |
| (VIIb, R = 2-thienyl, R′ = —CH═CH-2-methoxyphenyl) | >100 | uM |
| (VIIc, R = 2-thienyl, R′ = —CH═CH-2-nitrophenyl) | 12 | uM |
| (VIIr, R = 3-pyridyl, R′ = 2,3-dihydroxyphenyl | 29 | uM |
| (Xa, R = 2-thienyl, X = H and Ar = phenyl, R′ = Me) | 15 | uM |
| (Xf, R = methyl, X = H and Ar = phenyl, R′ = Me) | 17 | uM |
| (Xd, R = 2-furyl, X = H and Ar = phenyl, R′ = Me) | 14 | uM |

### Example 3

### Methods for Assessing Anticancer Activity

The primary in vitro screen for the anticancer activity described in this patent is based on the ability of the beneficial therapeutic to inhibit the proliferation of tumor cells grown in culture. Representative examples of the compounds of the present invention, were tested in this assay for biological activity and found to be potent inhibitors of cell growth in colon carcinoma (HT29 cells), in breast cancer (MCF-7 cells), in cervical cancer (HeLa cells) and in skin cancer (PAM 212 cells). These findings directly demonstrate that the newly synthesized compounds are potential therapeutics for human proliferative diseases. A description of this assay on PAM 212 cells as an example follows.

The ability of any one of the above compounds to inhibit cell growth is directly related to its therapeutic potential as an anticancer agent. This type of growth assay can be used with mammalian cancer cells as well as with pathogenic and non-pathogenic microbes including, but not limited to, yeasts and bacteria.

Typical growth inhibition curves for compounds of classes VII is shown in FIG. 3.

Typical growth inhibition curves for different cell lines for compound VIIo are shown in FIG. 4.

Members of triazole families VII and X displayed anticancer activity in this assay (PAM 212 cells). Typical values as IC₅₀'s obtained from the testing and graphical analysis described above were:

| | | |
|---|---|---|
| (VIIc, R = 2-thienyl, R′ = —CH═CH-2-nitrophenyl) | 60 | uM |
| (VIIe, R = 2-furyl, R′ = —CBr═CH—Ph) | 4.5 | uM |
| (VIIg, R = methyl, R′ = —CBr═CH—Ph) | 6 | uM |
| (VIIn, R = 2-thienyl, R′ = —C(CH₃)═CH—Ph) | 60 | uM |
| (VIIo, R = 2-thienyl, R′ = —CBr═CH—Ph) | 5 | uM |
| (Xa, R = 2-thienyl, X = H and Ar = phenyl, R′ = Me) | 44 | uM |
| (Xd, R = furyl, X = H and Ar = phenyl, R′ = Me) | 45 | uM |
| (Xf, R = methyl, X = H and Ar = phenyl, R′ = Me) | >100 | uM |
| (Xo, R = 2-thienyl, X = Br and Ar = phenyl, R′ = Me) | 0.4-0.5 | uM |
| (Xp, R = 2-thienyl, X = Cl and Ar = phenyl, R′ = Me) | 4 | uM |

Materials and Methods

Assessing Inhibitors of Nitric Oxide Synthase:

PAM 212 cells were maintained in growth medium consisting of Dulbecco's modified Eagles's medium (DMEM) supplemented with 10% fetal calf serum. For each assay, cells were inoculated into 24 well tissue culture plates (250,000 cells/well) in growth medium. After 24 hours, the medium was changed to phenol red and serum free DMEM containing 100 U/ml of gamma interferon. After 72 hours, nitric oxide production by the cells was quantified spectrophotometrically by measuring the accumulation of nitrite in the culture medium using the Greiss reagent. An aliquot of the culture medium was mixed with equal volumes of 1.0% sulfanilamide and 0.1% N-1-naphthylethylene diamine in 50% phosphoric acid. After 15 minutes at room temperature, the absorbance of the resulting chromophore was measured at 540 nm using a microplate reader and compared to standard solutions of sodium nitrite.

Assessing Anticancer Activity:

To assay these compounds for anticancer activity, tumor cells (PAM212) grown in vitro in monolayer culture were used. Cells were inoculated into 6 well culture dishes (3.5 cm diameter per well, 25,000 cells per well) in 2 mL of growth medium consisting of Dulbecco's modified Eagle's medium supplemented with 10% calf serum. After 24 hours at 37° C. in a humidified incubator with an atmosphere containing 5% carbon dioxide, the growth medium was drained from the cells and replaced with 2 mL of growth medium containing either control vehicle or increasing concentrations of the candidate anticancer agents. Triplicate wells on the plates were used to measure control growth and growth of the cells in the presence of each concentration of anticancer agent. The cells were then returned to the incubator. After the cells had grown for 4 to 5 days, the medium was drained from the culture dishes and the cells washed with phosphate buffered saline.

The cells from each well on the culture dishes were removed by trypsin treatment and counted with a Coulter counter. The assay may be performed on a variety of tumor cells in culture. As specific examples of this assay, cells were treated with increasing concentrations of the compounds indicated in the Table. After four days in culture, the number of cells in each well of the plate was determined. The data can be presented as a curve showing the inhibition of tumor cell growth with increasing concentrations of the compound. The concentration inhibiting cell growth by 50% (IC₅₀) is determined from the curve. This is shown in the table for a variety of anticancer therapeutics. Note that each of the compounds tested was a potent inhibitor of cell growth. The IC₅₀ values are typically in the micromolar concentration range.

### FIGURES

FIG. 1: Summary of Compounds of Interest: Provides chemical structure for VIIe, VIIn, VIIt, VIIo and VIIc.

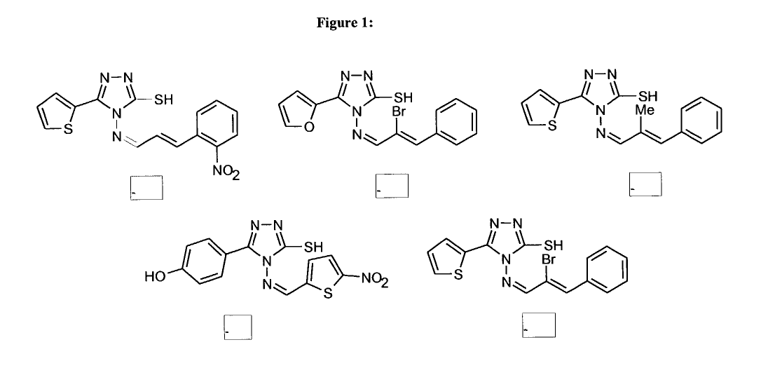

FIG. 2: A Typical Response Curve for Inhibitors of Nitric Oxide Synthase: Highlighting VIIa, R-2-thienyl, R′=CH═CH-Ph.

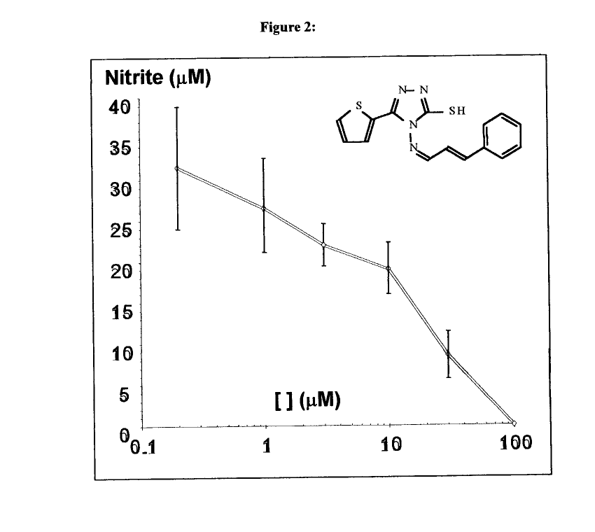

FIG. 3: Typical Growth Inhibition Curve: Highlighting class VII compounds.

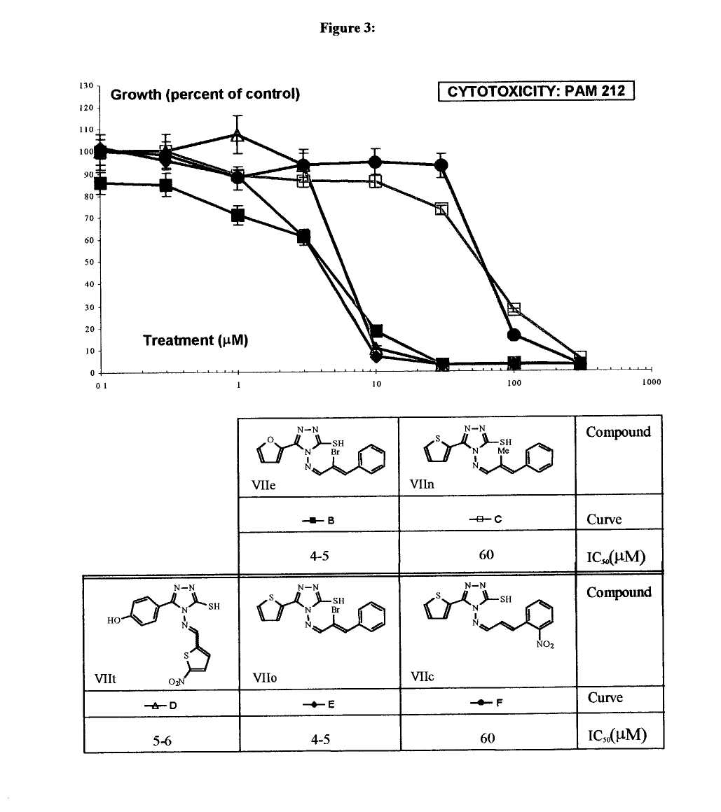

FIG. 4: Growth Inhibition Curve for VIIₒ: Against active colon cancer (HT29cells), breast cancer (MCF-7 cells), cervical cancer (HELA cells) and skin cancer (PAM 212 cells).

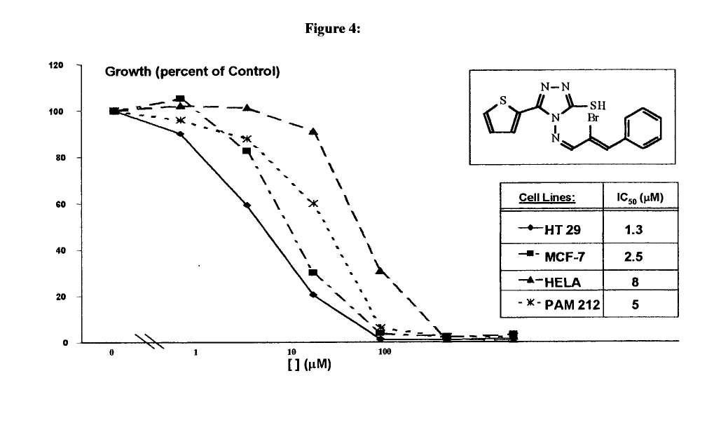

We claim the following:

1. A method for the inhibition of nitric oxide synthase in a subject in need of such inhibition wherein is administered an effective amount of an inhibitor having the Class of either VII or X as shown below. In each class, R and R′ may constitute alkyl, aryl, hydrogen, halogen, fluoroalkyl, or heterocyclic.

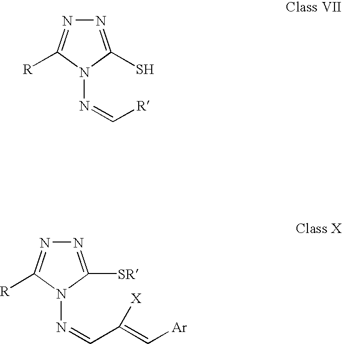

2. A method for the inhibition of cellular proliferation in a subject in need of such inhibition wherein is administered an effective amount of an inhibitor having the Class of either VII or X as shown below. In each class, R and R′ may constitute alkyl, aryl, hydrogen, fluoroalkyl, or heterocyclic.

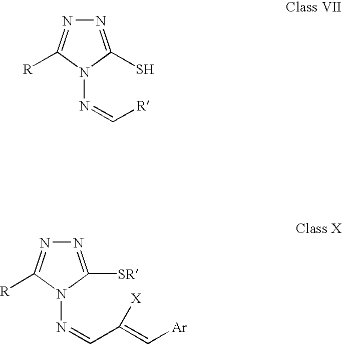

3. The use of compounds as in claim 2 for treatment of Cervical cancer.

4. The use of compounds as in claim 2 for treatment of Skin cancer.

5. The use of compounds as in claim 2 for treatment of Breast cancer.

6. A pharmacological assay method useful for detecting and ranking candidate drugs with nitric oxide synthase inhibition potential. This consists in quantification of suppression of NO release from PAM 212 keratinocytes stimulated to produce nitric oxide synthase by cytokine gamma interferon using either Class VII or X as shown in claim 1.

7. A pharmaceutical formulation comprising of a compound of Classes VII and X as defined in claim 1, or an optical isomer or racemate of any chiral analog thereof or a pharmaceutically acceptable salt thereof, optionally in a mixture with a pharmaceutically acceptable diluent or carrier.

8. A method for orally administering said compounds as in claim 7 in a pharmacologically acceptable carrier, said carrier including an ingredient selected from the group consisting of a binding agent, filler, lubricant, disintegrant, wetting agent, inert diluent, surface active agent, dispersing agent, suspending agent, emulsifying agent, edible oil, flavoring agent and mixtures thereof.

9. A method for the topically administering said compounds as in claim 7 in a pharmacologically acceptable carrier in the mouth, said carrier including an ingredient selected from the group consisting of a flavor, sucrose, acacia, tragacanth, gelatin, glycerin and mixtures thereof.

10. A method for nasally administering said compounds as in claim 7 in a pharmacologically acceptable carrier, said carrier including an ingredient selected from the group consisting of a dispersing agent, solubilizing agent, suspending agent and mixtures thereof.

11. A method for administering said compounds as in claim 7 in a pharmacologically acceptable carrier by inhalation, said carrier including a propellant.

12. A method wherein said propellant as in claim 7 is selected from the group consisting of dichlorodifluoromethane, trichlorofluoromethane, dichlorotetrafluoroethane, carbon dioxide and mixtures thereof.

13. A method for administering said compounds as in claim 7 in a pharmacologically acceptable carrier by inhalation or insufflation, said carrier including an ingredient selected from the group consisting of lactose, starch and mixtures thereof.

14. A method for administering said compounds as in claim 7 in a pharmacologically acceptable carrier parenterally, said carrier including an ingredient selected form the group consisting of an anti-oxidant, buffer, bacteriostat, suspending agent, thickening agent, saline, water and mixtures thereof.

15. A method of administering said compounds as in claim 7 in a pharmacologically acceptable carrier rectally, said carrier including an ingredient selected from the group consisting of cocoa butter, polyethylene glycol and mixtures thereof.

16. A method wherein said compounds as in claim 7 to be administered rectally includes an ingredient selected from the group consisting of an antimicrobial agent, an immunosuppressant, a preservative and mixtures thereof.

17. A pharmaceutical formulation comprising of a compound of Classes VII and X as defined in claim 2, or an optical isomer or racemate of any chiral analog thereof or a pharmaceutically acceptable salt thereof, optionally in a mixture with a pharmaceutically acceptable diluent or carrier.

18. A method for orally administering said compounds as in claim 17 in a pharmacologically acceptable carrier, said carrier including an ingredient selected from the group consisting of a binding agent, filler, lubricant, disintegrant, wetting agent, inert diluent, surface active agent, dispersing agent, suspending agent, emulsifying agent, edible oil, flavoring agent and mixtures thereof.

19. A method for the topically administering said compounds as in claim 17 in a pharmacologically acceptable carrier in the mouth, said carrier including an ingredient selected from the group consisting of a flavor, sucrose, acacia, tragacanth, gelatin, glycerin and mixtures thereof.

20. A method for nasally administering said compounds as in claim 17 in a pharmacologically acceptable carrier, said carrier including an ingredient selected from the group consisting of a dispersing agent, solubilizing agent, suspending agent and mixtures thereof.

21. A method for administering said compounds as in claim 17 in a pharmacologically acceptable carrier by inhalation, said carrier including a propellant.

22. A method wherein said propellant as in claim 17 is selected from the group consisting of dichlorodifluoromethane, trichlorofluoromethane, dichlorotetrafluoroethane, carbon dioxide and mixtures thereof.

23. A method for administering said compounds as in claim 17 in a pharmacologically acceptable carrier by inhalation or insufflation, said carrier including an ingredient selected from the group consisting of lactose, starch and mixtures thereof.

24. A method for administering said compounds as in claim 17 in a pharmacologically acceptable carrier parenterally, said carrier including an ingredient selected form the group consisting of an anti-oxidant, buffer, bacteriostat, suspending agent, thickening agent, saline, water and mixtures thereof.

25. A method of administering said compounds as in claim 17 in a pharmacologically acceptable carrier rectally, said carrier including an ingredient selected from the group consisting of cocoa butter, polyethylene glycol and mixtures thereof.

26. A method wherein said compounds as in claim 17 to be administered rectally includes an ingredient selected from the group consisting of an antimicrobial agent, an immunosuppressant, a preservative and mixtures thereof.

27. A method for treatment of cytotoxic or cytostatic organisms through the inhibition of nitric oxide synthase in a subject in need of such inhibition wherein is administered an effective amount of an inhibitor having the Class of either VII or X as shown below. In each class, R and R′ may constitute alkyl, aryl, hydrogen, halogen, fluoroalkyl, or heterocyclic.

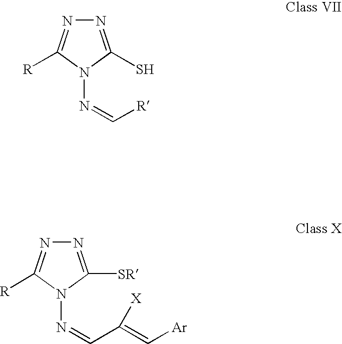

26. A method as in claim 26 to treat tuberculosis.

27. A method as in claim 26 to treat leprosy.

28. A method as in claim 26 to treat chronic inflammatory disorders of the gum including but not limited to periodontitis.

29. A method as in claim 26 to treat encephalomyelitis.

30. A method as in claim 26 to treat viral encephalitis.

31. A method as in claim 26 to treat autoimmune encephalitis.

32. A method as in claim 26 to treat dementia including but not limited to AIDS-related neurodegeneration.
